(19) United States
(12) Patent Application Publication
(10) Pub. No.: US 2009/0304639 A1
(43) Pub. Date: Dec. 10, 2009

(54) Method for preparing an organ for transplantation

(76) Inventors: Takashi Yokoo, Tokyo (JP); Masataka Okabe, Tokyo (JP); Tatsuo Hosoya, Tokyo (JP)

Correspondence Address: KILYK & BOWERSOX, P.L.L.C., 400 HOLIDAY COURT, SUITE 102, WARRENTON, VA, 20186, US

(21) Appl. No.: 11/919,317

(22) PCT Filed: Oct. 25, 2005
(86) PCT No.: PCT/JP2005/019552

§ 371 Date: Oct. 26, 2007

(30) Foreign Application Priority Data
Apr. 28, 2005 (JP) 2005-132811

(51) Int. Cl. A61K 45/00; A61P 43/00

(52) U.S. Cl. 424/93.7

(57) ABSTRACT

The present invention provides a means for achieving generation of a complex organ such as kidney and the like through the use of hMSCs to generate the human organ. The method for preparing a desired organ for transplantation to human by transplanting an isolated human mesenchymal stem cell to the embryo of a pregnant mammal host to induce differentiation of the mesenchymal stem cell is a method wherein the mesenchymal stem cell is transplanted into the embryo at a corresponding site for differentiation into the desired organ in the host at a transplantation time when the host is still at an immunologically tolerant stage.

### TECHNICAL FIELD

The present invention provides a method for preparing an organ for transplantation for humans.

### BACKGROUND ART

Organ regeneration has recently attracted considerable attention as a new therapeutic strategy. The potential for regenerative medicine has been gradually realized with the discovery of various tissue stem cells, and with reports of therapeutic benefits through the regeneration of neurons (non-patent document 1), β cells (non-patent document 2), myocytes (non-patent document 3), blood vessels (non-patent document 4) and the like. However, success using such strategies to date has been limited to the cells and simple tissues. Anatomically complicated organs such as the kidney and lung, which are comprised of several different cell types and have a sophisticated 3-dimensional organization and cellular communication, have proven more refractory to stem cell-based regenerative techniques.

With advances in medical transplantation, it is expected that these complex organs can be transplanted to bring complete recovery of a seriously damaged organ. However, there is a worldwide chronic shortage of donors. Furthermore, even successful transplantation needs a long-term administration of immunosuppressive drugs to avoid the rejection reaction, compelling recipients to continue suffering from the accompanying side-effects (non-patent document 5).

Therefore, one of the ultimate therapeutic aims is to establish self-organs from autologous tissue stem cells and transplant the in vitro-derived organ as a syngraft back into the donor individual.

Human mesenchymal stem cells (hMSCs) found in adult bone marrow has been recently made known to maintain plasticity and to differentiate into several different cell types, depending on their microenvironment (non-patent document 6). In contrast to embryonic stem cells (ES cells), hMSCs can be isolated from autologous bone marrow and applied for therapeutic use without any serious ethical issues or immunologic consequences (non-patent document 7).

[Non-patent document 1] J. Neurosci. Res. 69, 925-933 (2002)
[Non-patent document 2] Nat. Med. 6, 278-282 (2000).
[Non-patent document 3] Nature 410, 701-705 (2001)
[Non-patent document 4] Nat. Med. 5, 434-438 (1999)
[Non-patent document 5] Transplantation 77, S41-S43 (2004)
[Non-patent document 6] Science 276, 71-74 (1997)
[Non-patent document 7] Birth Defects Res. 69, 250-256 (2003)
[Non-patent document 8] Organogenesis of the Kidney (Cambridge Univ. Press, Cambridge, U.K.) (1987)
[Non-patent document 9] Exp. Nephrol. 10, 102-113 (2002)
[Non-patent document 10] Am. J. Kidney Dis. 31, 383-397 (1998)
[Non-patent document 11] J. Neurosci. Res. 60, 511-519 (2000)
[Non-patent document 12] Blood 98, 57-64 (2001)
[Non-patent document 13] J. Am. Soc. Nephrol. 11, 2330-2337 (2001)
[Non-patent document 14] Methods 24, 35-42 (2001)
[Non-patent document 15] J. Clin. Invest. 105, 868-873 (2000)
[Non-patent document 16] J. Neurol. Sci. 65, 169-177 (1984)
[Non-patent document 17] Kidney Int. 64, 102-109 (2003)
[Non-patent document 18] Cytometry 12, 291-301 (1991)
[Non-patent document 19] Dev. Growth Differ. 37, 123-132 (1995)
[Non-patent document 20] Am. J. Physiol. 279, F65-F76 (2000)
[Non-patent document 21] Eur. J. Physiol. 445, 321-330 (2002)
[Non-patent document 22] Proc. Natl. Acad. Sci. USA 97, 7515-7520 (2000)
[Non-patent document 23] Nature 418, 41-49 (2002)
[Non-patent document 23] Am. J. Physiol. 280, R1865-1869 (2001)

### DISCLOSURE OF THE INVENTION

### Problem to be Solved by the Invention

An object of the present invention is to provide a means for achieving the creation of a complex organ such as a kidney through a method to create a human organ through the use of hMSCs.

### Means for Solving the Problem

The organ of the present invention is not particularly limited, but as a representative target organ, the kidney was selected. It was because it represents a complex organ, comprising several different cell types, and having a sophisticated and three-dimensional organization, and its embryonic development has been well researched. Kidney development is initiated when the metanephric mesenchyme at the caudal portion of the nephrogenic cord (non-patent document 8) induces the nearby Wolffian duct to produce a ureteric bud (non-patent document 9). Development proceeds as a result of reciprocal epithelial-mesenchymal signaling between the ureteric bud and metanephric mesenchyme (non-patent document 10). To test whether hMSCs could participate in kidney development, hMSCs were initially cocultured with either rodent Wolffian duct extracted at the embryonic stage immediately before formation of the kidney primordia, or with established metanephric rudiment. However, this procedure was not sufficient to achieve kidney organogenesis or even integration of hMSCs into the developing rodent metanephros. This study suggests that hMSCs must be placed in a specific embryonic niche to allow for exposure to the repertoire of signals required to generate the organ. The present inventors discovered that this outcome can best be achieved by implanting hMSCs into the nephrogenic site of a developing embryo, and one of the present inventions was completed.

It is difficult to implant cells prenatally at the exact site of organogenesis by a transuterine approach. In addition, once embryos are removed for cell implantation, they cannot be returned to the uterus for further development. Therefore, the present inventors isolated embryos from uteri for cell implantation, after which the embryos were developed in vitro through whole-embryo culture until the embryos ended the initial stage of organogenesis, and further matured in organ culture and the abdominal cavity of a recipient. In the rest of the present invention, the present inventors find that by using this culture combination, hMSCs develop into morphologically identical cells to endogenous renal cells and are able to contribute to complex kidney structures. Furthermore, the present inventors show that this novel kidney has a filtering function and can receive the bloodstream from the recipient and generate urine, and the present invention was completed.

More specifically, the present invention includes:

1. A method for preparing a desired organ for transplantation to human by transplanting an isolated human mesenchymal stem cell to an embryo of a pregnant mammal host to induce differentiation of the mesenchymal stem cell, wherein the mesenchymal stem cell is transplanted to the embryo at a corresponding site for differentiation into the desired organ in the host at a time when the host is still at an immunologically tolerant stage.
2. The method according to item 1, wherein the desired organ is a kidney.
3. The method according to item 1, wherein the desired organ is a liver, pancreas, lung, heart, cornea, nerve, skin, hematopoietic stem cell or bone marrow.
4. The method according to any one of items 1 to 3, wherein the pregnant mammal host is a mammal having a similar size of the organ to that of the desired organ for human.
5. The method according to any one of items 1 to 3, wherein the pregnant mammal host is a pig.
6. The method according to item 5, wherein the mesenchymal stem cell is transplanted on a stage embryo day of 21 to 35.
7. The method according to any one of items 1 to 6, wherein the mesenchymal stem cell is transplanted to the embryo by transplanting the cell exactly to an organ-forming site of the host through a transuterine approach.
8. The method according to any one of items 1 to 6, wherein the mesenchymal stem cell is transplanted to the embryo by dissecting the embryo from the uterus and transplanting the cell exactly to an organ-forming site of the host, and then further growing the embryo in vitro using whole embryo culture.

### EFFECTS OF THE INVENTION

The present invention provides a novel means for autotransplantation of autologous organs. In other words, the isolated mesenchymal stem cells of an individual can be transplanted to an embryo inside a pregnant mammalian host at a desired site to induce differentiation into the desired organ, which is then transplanted to the individual.

### BRIEF DESCRIPTION OF THE DRAWINGS

FIG. 1-1 is a figure showing the ex utero differentiation of kidney primordia using the relay culture system. From the upper left column, the embryos, E11.5, E12, E12.5, E13, and E13.5 are shown, and in lower column, there are shown E11.5 embryos separated ex utero which are cultured 24 hours (left) and 48 hours (right) in whole-embryo culture containers.

FIG. 1-2 is a figure showing ex utero differentiation of kidney primordia using a relay culture system. To confirm the extent of tubulogenesis and enlarged ureteric bud branching, hematoxylin/eosin staining (b) and whole-mount in situ hybridization for c-ret (c) are shown.

FIG. 2-1 is a figure showing the proportion of donor-derived cells in the metanephros regenerated from hMSCs without genetic manipulation. M is the large informative peak.

FIG. 2-2 is a figure showing the proportion of donor-derived cells in the metanephros regenerated from hMSCs transfected with GDNF. M is the large informative peak.

FIG. 2-3 is a figure showing the assessment of DNA-ploidy of the regenerated donor-derived cells. M is the large informative peak.

FIG. 3-1 is a figure showing the differentiation of transplanted hMSCs into organized, resident renal cells. (a) After relay culturing, the resulting metanephros was subjected to an X-gal assay to trace the transplanted hMSCs.

FIG. 3-2 is a figure showing the differentiation of transplanted hMSCs into organized, resident renal cells. (b) Serial sections were examined by light microscopy. (c) Tissue sections were subjected to two-color immunofluorescent staining for beta-gal (left) and WT-1 (right).

FIG. 3-3 is a figure showing the differentiation of transplanted hMSCs into organized, resident renal cells. (d) After relay culturing, the resulting metanephros were treated with collagenase, and single cells were subjected to the FACS-Gal assay. LacZ-positive cells were sorted and, after RNA extraction, subjected to RT-PCR analysis. From the top, Kir6.1, SUR2, AQP-1, PTH receptor 1, 1 alpha hydroxylase, NBC-1, nephrin, podocine, GLEPP1, human-specific beta 2 microgloblin (MG) and rat GAPDH are shown.

FIG. 4 is a figure showing the injection and culture of hMSCs in isolated metanephros. (a) After 6 days of organ culture, the resulting metanephroses were subjected to an X-gal assay. (b) RNAs were extracted from LacZ-positive cells and subjected to RT-PCR. From the top, AQP-1, PTH receptor 1, NBC-1, GLEPP1, nephrin, podocine, rat GAPDH, and human-specific beta 2 microgloblin (MG) are shown.

FIG. 5-1 shows a therapeutic kidney regeneration in an alpha-gal A-deletion Fabry mouse. (a) The alpha-gal A enzymatic bioactivity of resulting metanephros was fluorometrically assessed.

FIG. 5-2 shows a therapeutic kidney regeneration in an alpha-gal A-deletion Fabry mouse. (b) To confirm the potency of the Gb3 clearance in resulting metanephros, organ culture was performed in the presence of Gb3, and accumulation in the metanephros was assessed by immunostaining for Gb3.

FIG. 6 is a figure showing the emergence of the metanephros transplanted in the greater omentum.

FIG. 7 is a figure showing the histological analysis of the metanephros (2 weeks) transplanted inside the greater omentum.

FIG. 8 is a figure showing transplantation (2 weeks) of different stages of kidney primordias to the greater omentum.

FIG. 9-1 is a figure showing the new kidney generated from hMSCs with improved relay culture (2 weeks)

FIG. 9-2 is a figure showing the histological findings of the new kidney created by the improved relay culture (2 weeks) in LacZ rat from LacZ positive human mesenchymal stem cells. It is shown that the glomerular epithelial cells (lower left) and the tubular epithelial cells (lower right) are derived from the injected hMSCs.

FIG. 9-3 is a figure showing the new kidney isolated, the hMSC-derived cells isolated by FACS-Gel assay, the RNA extracted, and then the gene expression analyzed by RT-PCR. The gene expressions for aquaporin-1 (AQP-1), parathyroid hormone (PTH) receptor 1, 1α hydroxylase, nephrin, glomerular epithelial protein 1 (GLEPP-1) and human-specific β2 microgroblin (MG) are shown. Lane 1 is the marker (φX174/HaeIII), lane 2 is hMSCs, and lanes 3-5 are new kidneys resulting from their respective experiments.

FIG. 9-4 is a figure showing an electron microscope photograph of the new kidney transplanted into the greater omentum. It is shown that red blood cells are seen in the glomerular loop and integrated with the recipient bloodstream.

FIG. 10-1 is a figure showing that, by using a LacZ transgenic rat as the recipient, the vascular system inside the new kidney is constructed from the recipient.

FIG. 10-2 is a figure showing the gene expression in the LacZ positive cells of intercellular adhesion molecule-1 (ICAM-1), vascular cell adhesion molecule-1 (VCAM-1), platelet endothelial cell adhesion molecule-1 (PECAM-1), and rat GAPDH. Lane 1 is marker φX174/HaeIII), Lane 2 is the kidney primordia immediately before transplanting into the greater omentum, and lanes 3-5 are the RNAs from the new kidneys resulting from their respective experiments.

FIG. 11 is a figure showing that the improved relay culture method (4 weeks) produces urine to form hydronephrosis (left), and the liquid accumulated in the expanded ureter (upper right) has a urine composition (lower right).

### DESCRIPTION OF THE PREFERRED EMBODIMENT

The present invention is an improvement of the method for preparing a desired organ for human transplantation by transplanting isolated human mesenchymal cells (hMSCs) to an organ inside a pregnant mammalian host to induce differentiation of hMSCs.

The suitable example of a mammal which can be used in the present invention is a pig. Other suitable animals include genemanipulated pigs such as transgenic, knockout, or knock-in pigs. Other examples include ungulates such as cow, sheep, pig, goat, and horse. Further suitable examples include genetically modified animals, particularly transgenic animals of mouse or the aforementioned ungulates.

The hMSCs is isolated from human bone marrow. The isolation is performed by a general surgical procedure. The isolated cells are cultured under a selected optimal condition, but the passage number is preferably 2-5 or less. The culture medium kit for human mesenchymal stem cells manufactured by Cambrex Bio Science is used to keep culturing the hMSCs as it is not transfected.

If desired, the cell is transfected with a desired gene by the manipulation using, for example, adenovirus and/or retrovirus. For example, if a kidney is desired to provide, the cell is transfected with a gene in order to express the glial cell-line derived neurotrophic factor GDNF for assisting formation of the kidney. This is because the transfection facilitates the mesenchymal tissue to express GDNF immediately before kidney formation so that the ureteric bud expressing the c-ret, a receptor for the factor is taken into the process to complete the first important step for kidney generation. This transfection is confirmed to raise the formation rate of an injected stem cell-derived kidney from 5.0±4.2% to 29.8±9.2%.

The prepared hMSCs is then transplanted to an embryo inside a pregnant mammalian host animal. From a technical reason, the embryos may be dissected from the host body to develop by so-called whole embryo culture, but the cells are more preferably transplanted directly to the embryo inside the body to form the organ inside the uterus. The transplantation is performed by general surgical methods, for example, using a micropipette while examining under echo. 0.5−1.0×10³ of the cells are sufficient for transplantation.

The timing for transplantation to the embryo may be optionally decided. In the experiments using rats, the embryonic stage day of 11.5 was suitable. Even in a large mammal such as pig, the similar embryonic stage is suitable. However, by selecting appropriate conditions, an earlier or later stage may also be selected. In any case, it is important that the cells should be transplanted to the embryo at least at a time when the host is still at an immunologically tolerant stage.

The characteristic of the present invention is to select a transplantation site. In other words, the transplantation site for the hMSCs to the embryo is a corresponding site for generation of the desired organ in the host. Therefore, the cells must be transplanted at a site at a time when the site can be confirmed to be a corresponding site for the desired organ. However, the bud cells for the desired organ must be in a sprouting state prior to starting development. For example, if a kidney is desired, the site is a sprouting site of the ureteric bud. Or else, for a liver, the site is a developing site of the liver bud (liver diverticulum) formed as a protrusion from the tail end of the foregut to the abdominal side. In addition, if a pancreas is desired, the cell is injected at a developing site of the pancreatic bud generated in the foregut from the tail side.

If the cells are developed ex vivo, the embryo is cultured through so-called whole-embryo culture (uteri are dissected from mothers, and embryos are freed from uterine wall, decidua, and the outer-membrane layer including Reichert's membrane, and then transplanted with human mesenchymal stem cells, and then cultured in a culture bottle or the like). After a certain development, the embryo is assessed in morphology and function, and the organ primordia are confirmed. And then the organ primordia are separated to subject to organ culture.

If the cells are developed in vivo, the human mesenchymal stem cells are directly transplanted by a transuterine approach to an embryo inside the live body of a large pregnant mammal such as a pig, and left to grow inside the living body into an organ.

There are many organs to which the present invention can be adapted. Suitable examples include, but not limited to, liver, pancreas, lung, heart, cornea, nerve, skin, hematopoietic stem cell, or bone marrow. Since the obtained organ has a homologous size to the organ of the host animal, the host is preferably a mammal having a similar size of organ to the desired organ of human in order to exhibit adequate function in human. However, the host has not necessarily exactly the same size of the organ. For example, even an obtained kidney, which has as little as 1/10^{th} of the perfect function, works adequately to allow exemption from burdensome dialysis. Even an obtained liver, which has as little as ⅕^{th} of the perfect function, is sufficient to allow supporting life. For this reason, pigs are the optimal hosts, and even miniature pigs have adequate size of organs to exhibit the function in human.

The organ thus grown is, after confirmation of its function, is then dissected from the host, and returned to the human body. The organ is transplanted in the greater omentum of a human body as one of preferred sites. The transplanted kidney continues developing in the body, and acquires a suitable urinary excretion system to complete formation of a cloned kidney which exhibits renal function.

In order to exempt the formed organ from contamination with antigenic substances from the host, transformation of transplanted cells as follows is effective. Namely, the formed organ contains a coexistence of hMSCs-derived human cells and the host animal-derived cells. When the organ is transplanted to the human, the host-derived cells in coexistence are likely to trigger an immunological rejection reaction. Therefore, the formed organ must be completely cleared of the host-derived cells. In order to solve this problem, the host animal is designed to induce controllable programmed cell death and then allowed to form the desired organ. The embryo of the host animal is transplanted with the hMSCs at a corresponding site to form a desired organ, which is then allowed to induce cell death specific to the host cell, thereby to clear completely of the host-derived cells in a step prior to transplantation into human.

### Examples

As a representative example of the present invention, a system for a kidney using rat will be described. The present invention is not limited to this system, but includes all systems in which the hMSCs is used at a site and at a time selected for transplantation.

### (Materials and Methods)

1) Experimental Animals The animals used were wild-type Sprague-Dawley rats which were purchased from Sankyo Lab Service (Tokyo). At the Laboratory Animal Center of the Jikei University School of Medicine, a breeding colony of Fabry mice was established from breeding pairs donated by Mr. R. O. Brady (National Institute of Health, Bethesda). The midpoint at which a vaginal plug was seen was designated as day 0.5. Animals were housed in a ventilated (positive pressure airflow) rack and were bred and raised under pathogen-free conditions. All experimental procedures were approved by the Committee for Animal Experiments of the Jikei University School of Medicine.
2) Culture and Manipulation of hMSCs

hMSCs obtained from the bone marrows of healthy volunteers were used. Bone marrow-derived hMSCs that were confirmed to be CD105-, CD166-, CD29-, CD44-positive, and CD14-, CD34-, CD45-negative were purchased from Cambrex Bio Science Co. (Walkersville, Md.). Following the protocol provided by the manufacturer, these were cultured. In order to avoid phenotypic changes, the hMSCs were used within five cell passages. A replication-defective recombinant adenovirus carrying human glial cell line-derived neurotrophic factor GDNFcDNA (AxCAhGDNF) was generated and purified as described (non Patent document 11). Packaging cells (Ψ-crip) that produce a recombinant retrovirus bearing the bacterial LacZ gene (MFG-LacZ) were donated by H. Hamada (Sapporo Medical University, Sapporo, Japan). Adenoviral and retroviral infection were performed as described (non-patent documents 12, 13). The cells were labeled with 1, 1′-dioctadecyl-3,3,3′,3′-tetramethylindocarbocyanine (DiI) (Molecular Probes) at 0.25% (wt/vol) in 100% dimethylformamide and injected by using micropipettes at the sprouting site of ureteric bud.

### 3) Whole-Embryo Culture and Organ Culture

Whole embryos were cultured in vitro according to a previously described method (non-patent document 14, with several modifications. Using a stereoscopic microscope, uteri were dissected from anaesthetized mothers. Stage embryonic day (E) 11.5 rat embryos and stage E9.5 mouse embryos were freed from the uterine wall, decidua, and the outer-membrane layer, including Reichert's membrane. The yolk sac and amnion were opened to enable injection, but the chorioallantoic placenta was left intact. Successfully injected embryos were immediately cultivated in 15-ml culture bottles containing 3 ml of culture media consisting of 100% centrifuged rat serum supplemented with glucose (10 mg/ml), penicillin G (100 units/ml), streptomycin (100 micrograms/ml), and amphotericin B (0.25 micrograms/ml). The culture bottles were allowed to rotate in an incubator (model no. RKI10-0310, Ikemoto, Tokyo). Ex vivo development of the rat embryos was assessed after 24- and 48-hour culture periods and compared with E12.5 and E13.5 rat embryos. Forty-eight hours later, embryos were assessed for heartbeat, whole-body blood circulation, and general morphology. Kidney primordias were dissected and cultured as described previously (non-patent document 15). To enhance the accumulation of globotriaosylceramide (Gb3) in the kidney primordia, the cultivated metanephros were cultured in the presence of ceramide trihexoside (1 nmol, Sigma) (non-patent document 16). Alpha-galactosidase A (alpha-gal A) enzymatic activity in metanephros was fluorometrically assessed as described (non-patent document 17).

### 4) Histology

Two-color staining of metanephros was performed essentially as described (non-patent document 17) by using mouse anti-beta-gal (Promega) and rabbit anti-human WT-1 (Santa Cruz Biotechnology) as the primary antibodies. A monoclonal mouse anti-Gb3 antibody (Seikagaku, Tokyo) was also used. Whole-mount in situ hybridization with digoxigenin UTP-labeled c-ret riboprobes was performed as described (non-patent document 15). In situ hybridization was also performed on histological sections by using biotin-labeled human genomic AluI/II probes (Invitrogen) according to the manufacturer's protocol. An X-gal assay was used to assess expression of the LacZ gene as described (non-patent document 13).

### (X-Gal Assay)

The kidney, which differentiated in the greater omentum for 2-4 weeks, was fixed with PBS which contains 0.25% glutaraldehyde and 2% PFA (paraformaldehyde) for 3 hours at 4° C., and was washed three times for twenty minutes each at room temperature with wash buffer solution (0.02% NP-40, 0.01% deoxycholate in PBS). This was incubated for 3 hours at 37 degrees C. in a reaction buffer solution containing 1 mg/ml of X-gal(4-Cl-5-Br-3-indolyl-β-galactosidase), 5 mM potassium ferrocyanide (Sigma), 0.002% NP-40, 0.001% deoxycholic acid, and 2 mM MgCl₂. The entire kidney was then fixed in formalin and immersed in paraffin. Three micrometer sections were cut, and the counter (not an object) was stained with eosin, and LacZ positive cells were stained blue.

5) Identification of hMSC-Derived LacZ-Positive Cells

Metanephros generated by relay culture were digested in 500 microliters of collagenase type I (1 mg/ml) for 30 min at 37° C. 10% FBS (fetal bovine serum)-containing DMEM was added, and the cells were pelletized. Cell digestion products were filtered with a sterile double layered 40 micrometer nylon mesh and labeled with fluorescein digalactoside (FDG) (Molecular Probes) by making use of transient permeabilization through hypotonic shock (non-patent document 18).

### (FACS-Gal Assay)

In summary, cells were suspended at a concentration of 0 in 100 microliters of 4% FBS-containing PBS and heated to 37° C. An equal amount of 2 mM/L concentration of FDG in water was also heated to 37° C. The preheated cells and FDG were rapidly mixed and immediately returned to a water bath and left for 1 minute. 1.5 micromolar propidium iodide-containing 1.8 mL of ice-cold PBS was added. Thereupon, LacZ positive cells were sorted using a cell sorter (Becton Dickinson). Total RNA was extracted and subjected to RT-PCR to analyze expression of aquaporin-1 (AQP-1), parathyroid hormone (PTH) receptor 1, 1 alpha hydroxylase, Na⁺-HCO₃⁻ co-transporter 1 (NBC-1), nephrin, podocine, and glomerular epithelial protein 1 (GLEPP-1). For the analysis of cell ploidy, cells were stained with propidium iodide, and DNA content was assessed by using a flow cytometer.

### (RT-PCR)

Total RNA was extracted from LacZ-positive cells with RNeasy mini kit (Qiagen GnbH, Hilden Germany). Using Superscript II Reverse Transcriptase (Life Technologies BRL, Rockville, Md.), cDNA was synthesized following the protocol in the accompanying document. After PCR, the amplification product was analyzed for aquaporin-1 (AQP-1), parathyroid hormone (PTH) receptor 1, 1α hydroxylase, nephrin, glomerular epithelial protein 1 (GLEPP-1), intercellular adhesion molecule-1 (ICAM-1), vascular cell adhesion molecule-1(VCAM-1), and platelet-endothelial cell adhesion molecule-1(PECAM-1). A list of primer sequences and reaction conditions used can be found in Table 1. For human MG and rat GDPDH, a two-step amplification (43 cycles of 1 minute at 94° C., 1 minute at 66° C.) was used. PCR conditions were 36 cycles of (10 minutes at 95° C.-45 seconds at 94° C., one minute at optimum annealing temperature, 1 minute at 72° C.), and 10 minutes at 72° C.

TABLE 1

| | | | |
|---|---|---|---|
| Primer | Base sequence | Sequence length bp | Optimum temperature |

| | | | |
|---|---|---|---|
| Human 1α hydroxylase sense | CCTGAACAACGTAGTCTGCG | 620 | 60 |
| Human 1α hydroxylase antisense | CAGCTGTGATCTCTGAGTGG | | |
| Rat ICAM-1 sense | CTGGAGAGCACAAACAGCAGAG | 385 | 55 |
| Rat ICAM-1 antisense | AAGGCCGCAGAGCAAAAGAAGC | | |
| Rat VCAM-1 sense | TAAGTTACACAGCAGTCAAATGGA | 283 | 50 |
| Rat VCAM-1 antisense | CACATACATAAATGCCGGAATCTT | | |
| Rat PECAM-1 sense | AGGGCTCATTGCGGTGGTTGTCAT | 348 | 52 |
| Rat PECAM-1 antisense | TAAGGGTGCCTTCCGTTCTAGAGT | | |
| Human AQP-1 sense | CTTGGACACCTCCTGGCTATTGAC | 625 | 60 |
| Human AQP-1 antisense | AGCAGGTGGGTCCCTTTCTTTCAC | | |
| Human PTHs sense | GATGCAGATGACGTCATGAC | 482 | 58 |
| Human PTHs antisense | CAGGCGGTCAAACACCTCCCG | | |
| Human GLEPP-1 sense | TCACTGTGGAGATGATTTCAGAGG | 74 | 58 |
| Human GLEPP-1 antisense | CGTCAGCATAGTTGATCCGGA | | |
| Human nepbrio sense | CAACTGGGAGAGACTGGGAGAA | 168 | 56 |
| Human nepbrio antisense | AATCTGACAACAAGACGGAGCA | | |
| Human β-microgloblin sense | CAGGTTTACTCACGTCATCCAGC | 235 | ″ |
| Human β-microgloblin antisense | TCACATGGTTCACACGGCAGG | | |
| Rat GDPDH sense | CATCAACGACCCCTTCATT | 197 | ″ |
| Rat GDPDH antisense | ACTCCACGACATAGTCAGCAC | | |

### 6) Creation of Functional Donor-Derived Clone Kidney

In order to study the optimal conditions for the growth of the kidney primordia inside the greater omentum, the degree of growth after implantation was evaluated by the growth stage of the rat metanephros tissue and presence or absence of heminephrectomy. In accordance with the optimal conditions, the kidney primordia created as described above were implanted into the greater omentum of the recipient. After 2 weeks, whether there were findings of highly differentiated tissues of the kidney was confirmed by immunologic staining and electron microscopy.

7) Confirmation of the Integration Between the Blood Vessels of the Recipient and Clone Kidney

In order to confirm that there was blood flow of the recipient to the new kidney, the kidney was transplanted to the greater omentum of LacZ transgenic rat. It was confirmed that the blood vessels inside the new kidney were derived from the recipient. The human mesenchymal stem cells further injected also introduced the LacZ gene, and whether the blood vessels and the donor derived-nephrons were integrated, was confirmed.

### 8) Confirmation of the Presence or Absence of Urine Generating Function

In order to study whether the new kidney, which was grown in the greater omentum and had circulation of recipient's blood, can filter the recipient blood and generate urine, the kidney was developed for 4 weeks inside the greater omentum, and the urea nitrogen concentration and creatinine concentration inside the liquid collected in the ureter were measured and compared with the serum concentration to confirm the presence or absence of urine generating capability.

### 9) Statistical Analysis

Data were expressed as the mean±standard deviation. Statistical analysis was performed by using the two-sample t test to compare data in different 2 groups. P<0.05 was taken to be statistically significant.

### (Results)

### A. Ex-Utero Development of Kidney Primordia by Using the Relay Culture System

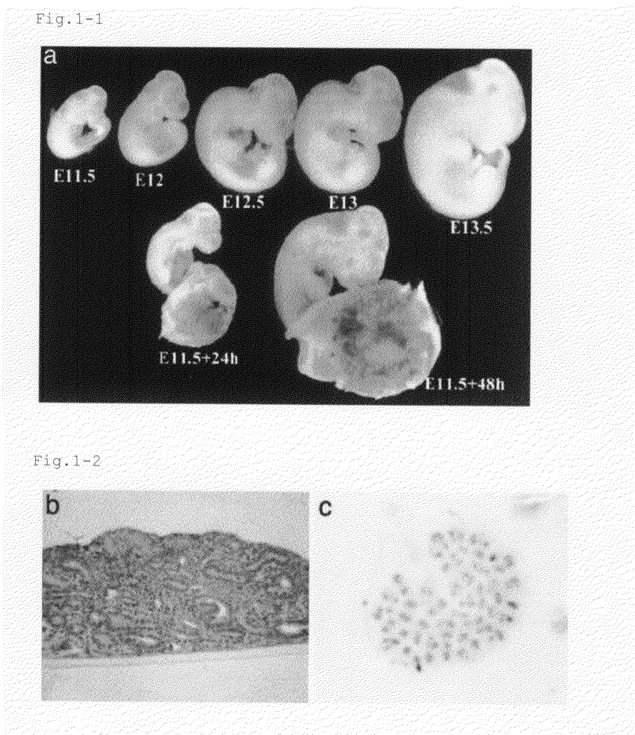

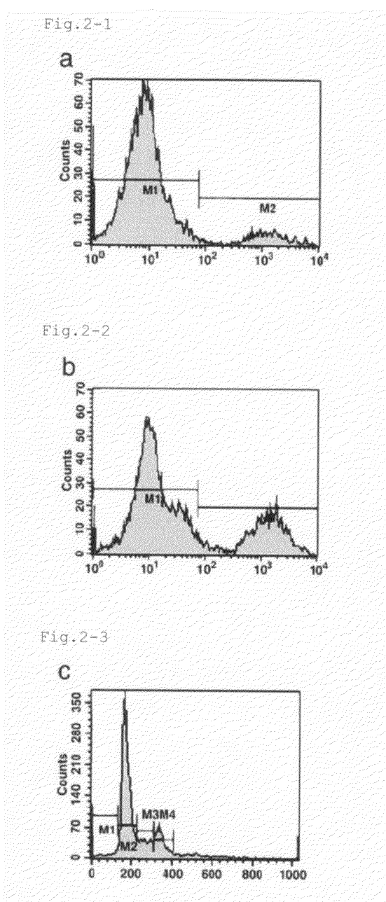

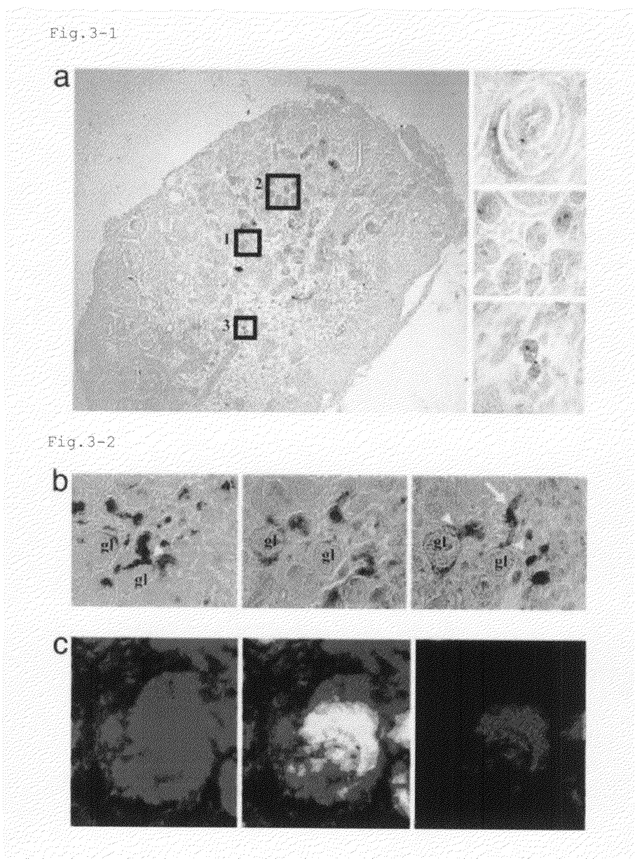

The whole-embryo culture system was optimized to allow a defined concentration of oxygen to be supplied continuously to rotating culture bottles, thus improving embryonic development ex utero (non-patent document 14). Using this system, rat embryos (E11.5) were cultured at 37° C. in the culture bottle consisting of the media composed of 100% freshly centrifuged rat serum supplemented with glucose (10 mg/ml), together with the yolk sac, amnion, and chorioallantoic placenta. After 24 and 48 hours in culture, ex utero development of the rat embryos was assessed by comparing with those that grown in utero for E11.5, E12.0, E12.5, E13.0, and E13.5. Forty-eight hours later, embryos were assessed for heartbeat, whole-body blood circulation, and general morphology. Based on the resultant somite number and general morphology, the developmental age of rat embryos cultured in this way appeared consistent with E 13 embryos that had developed in utero (FIG. 1-1(a)). At this stage, ureteric buds were elongated and initial branching was completed, indicating that during culture, the metanephric mesenchyme had been stimulated to take the first step toward nephrogenesis. However, embryos could not develop further and died soon after 48 hours because of insufficient development of the placenta in vitro (non-patent document 19). To overcome this limitation, whole-embryo culture was followed by organ culture. After whole-embryo culture for 48 hours, metanephros were dissected from embryos and subjected to organ culture for 6 days. Using this combination, which will be referred to as relay culture, kidney primordias continued to differentiate and grow in vitro. Repeated tubule formation and ureteric bud branching were confirmed by performing hematoxylin/eosin staining (FIG. 1-2(b)) and whole-mount in situ hybridization for c-ret (FIG. 1-2(c)). This shows that the metanephros can complete development ex utero, even if the embryo is dissected from the uterus before the stage at which the ureteric bud sprouts.

### B. Proportion of Donor-Derived Cells in Culture-Derived Metanephros and Assessment of the Possibility of Cell Fusion

Using the system described in A, hMSCs were injected into rat embryos at the kidney-forming site. In order to distinguish from the host-derived cells, the hMSCs was forced to express the LacZ gene using retrovirus, and the hMSCs labeled with DiI fluorescent were injected into the budding site of the ureteric bud of the rat embryo using adenovirus transfected with GDNF (FIG. 2-2(b)) or without (FIG. 2-1(a)). A total of 1×10³/embryo of hMSCs were then injected into the intermediate mesoderm between the somite and the lateral plate at the level of somite 29 for rat and somite 26 for mouse. The present inventors previously estimated these levels, by in situ hybridization for c-ret, to be the ureteric budding sites (non-patent document 15). Successful injection was confirmed by the fact that injected hMSCs-derived cells were detected along the Wolffian duct by in situ hybridization for human genomic AluI/II which identifies exclusively human cells.

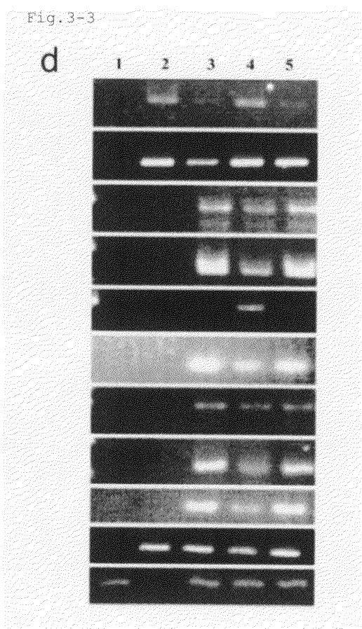

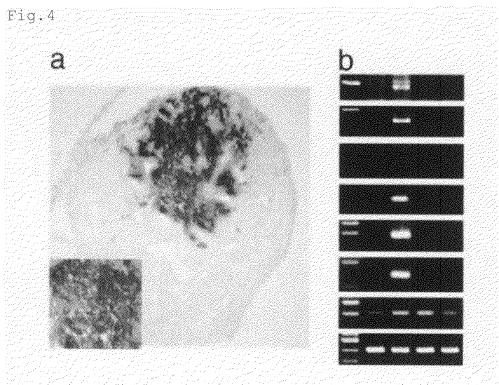

After relay culture, the newly generated kidney primordia was digested with collagenase, and when single cells were subjected to FACS-Gal assay, LacZ-positive cells were detected in the kidney primordia (5.0±4.2%) (FIG. 2-1(a)). No LacZ positive cells were detected in the isolated metanephros when the injection site was altered by over 1 somite in length. In control embryos, injection of labeled mouse fibroblasts instead of hMSCs resulted in a almost negligible number of LacZ-positive cells detected. To enhance the number of injected donor-derived cells, the hMSCs before injection were further modified to temporally express GDNF by using the adenovirus AxCAh-GDNF (Non-patent document 11). This is because GDNF is normally expressed in metanephric mesenchyme at this stage, and through the interaction between GDNF and its receptor, c-ret, epithelial-mesenchymal signaling is essential for the kidney formation (Non-patent document 10). The FACS-galactosidase assay revealed a significant increase in the number of donor-derived LacZ-positive cells detected in the kidney through this transient GDNF expression (29.8±9.2%, FIG. 2-2(b)). When LacZ-positive cells were sorted, and their DNA content was assessed by using propidium iodide intensity, 68.8±11.4% of LacZ-positive cells in the neogenerated kidney primordia was euploid (FIG. 2-3(c)). In addition, the number of LacZ-positive cells was significantly increased (2.84±0.49×10⁵/kidney primordia) compared with the starting number of injected cells (1×10³/embryo), suggesting that the remaining polyploid cells were mostly undergoing cell division. Furthermore, fluorescent in situ hybridization using the human and rat Y chromosome showed no cells that were doubly positive for the Y chromosome. These data strongly suggest that it is extremely unlikely that there will be cell fusion of host cell and donor cell.

C. Differentiation of Transplanted hMSCs into Kidney Cells

After relay culturing, the migration and morphologic changes of the hMSCs transplanted in the resulting kidney primordia were traced. In the organ culture, when the kidney primordia during growth was observed over time under a fluorescent microscope, DiI-positive hMSCs migrated towards the medulla, and an image of these cells dispersing in the kidney primordia was confirmed. In order to study whether these cells contribute to renal structures, the kidney primordia was subjected to an X-gal assay. LacZ-positive cells were scattered throughout the metanephric rudiment and were morphologically identical to glomerular epithelial cells (upper right), renal tubular epithelial cells (right center), and interstitial cells (lower right) (FIG. 3-1(a)). Furthermore, examination of serial sections of kidney primordia under a light microscope showed glomerular epithelial cells linked to tubular epithelial cells (arrow), and some of these cells formed a continuous tubular extension toward the medulla (arrow) (FIG. 3-2(b), gl: glomerulus). This image not only shows that, after transplantation, the hMSC differentiates into individual kidney cells, but also shows the formation of nephrons (the basic unit for filtration and reabsorption). For further confirmation of differentiation into glomerular epithelial cells, two-color immunofluorescent staining for beta-gal (left) and WT-1 (right) was conducted. WT-1 is known to be strongly expressed in glomerular epithelial cells at this stage (non-patent document 20). Because there were cells that were positive for both (center), this shows that some of LacZ-positive donor cells has completed differentiation to glomerular epithelial cells (FIG. 3-2(c)).

After relay culture, the resulting kidney primordia were digested, and single cells were subjected to the FACS-galactosidase assay. LacZ-positive cells were sorted and subjected to RT-PCR for expression analysis of Kir6.1, SUR2, AQP-1, PTH receptor 1, 1 alpha hydroxylase, NBC-1, nephrin, podocine, GLEPP1, human-specific beta 2 microglobin (MG), and rat GAPDH. Lane 1 is the control rat metanephros, lane 2 is hMSCs, and lanes 3-5 are the kidneys formed from three individual experiments. It was shown that donor-derived LacZ-positive cells expressed glomerular epithelial cell-specific genes (nephrin, podocine, and GLEPP-1) and renal tubular epithelial cell-specific genes (AQP-1, 1 alpha hydroxylase, PTH receptor 1, and NBC-1) (FIG. 3-3(d)). In contrast to endogenous renal cells, ATP-sensitive K⁺ channel subunit, Kir6.1/SUR2 (non-patent document 21) expressed in hMSCs was still expressed after relay culture.

D. Injection and Culture of hMSCs in Isolated Metanephros

hMSCs which express the LacZ gene through the use of retrovirus were further transfected with GDNF by adenovirus and injected into the cultured metanephros (E13). After 6 days of organ culture, the resulting metanephros were subjected to an X-gal assay (FIG. 4(a)). The inset shows LacZ positive cells at high magnification. The injected hMSCs-derived cells remain aggregated and do not form high-dimensional kidney structures. After sorting the LacZ positive cells, RNAs were extracted and subjected to RT-PCR. Neogenerated kidney before (lane 2) and after (lane 3) organ culturing is shown. Mixture of metanephros and hMSCs before (lane 4) and after (lane 5) organ culture is shown. Lane 1 is a marker (φX174/HaeIII). As shown in the figure, when hMSCs is injected into culture tissue which has already differentiated to metanephros, kidney-specific genes are not expressed (FIG. 4(b)). From the above items, only hMSCs which were injected before the sprouting of ureteric buds could integrate with the kidney primordias in the organ culture and be transformed to express kidney-specific genes. The gene expression capability can not be achieved under other conditions. In other words, the above shows that during whole-embryo culture, hMSCs complete an initial step essential for commitment to a renal fate and that during organ culture, they further undergo a mesenchyme-to-epithelium transition or stromogenic differentiation.

### E. Therapeutic Kidney Regeneration in α-Gal A Deletion Fabry Mice

To examine whether the hMSCs-derived nephron is functional, hMSCs were transplanted to an E9.5 embryo of a knockout mouse which does not express the α-gal A gene (Fabry mouse) and a relay culture was carried out (non-patent document 22). This deletion of α-gal is known in human as Fabry disease causing mainly an abnormal accumulation of sphingoglycolipid (Gb3) in the glomerular epithelial cells and renal tubular epithelial cells, and kidney disorder after birth.

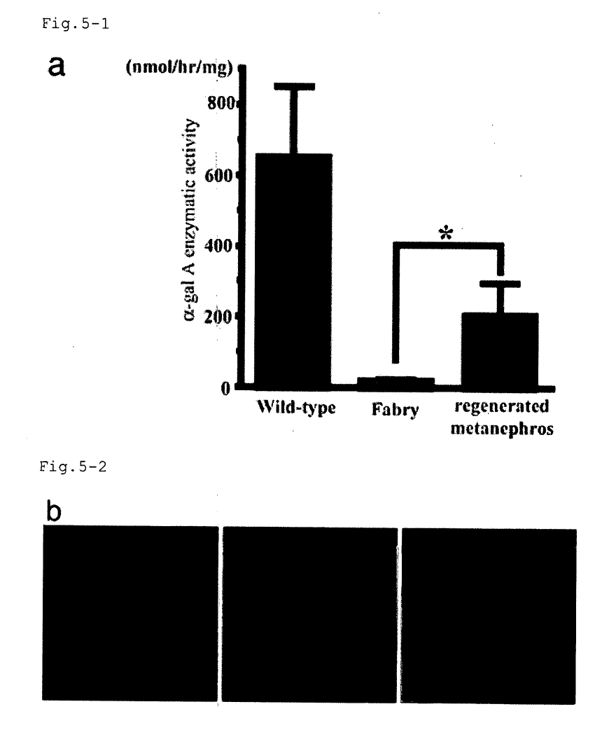

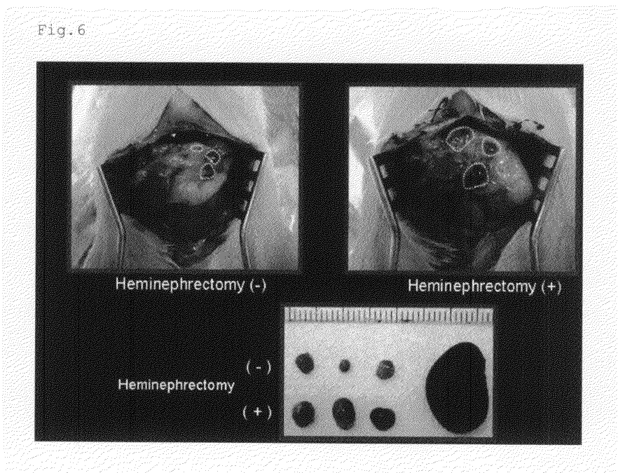

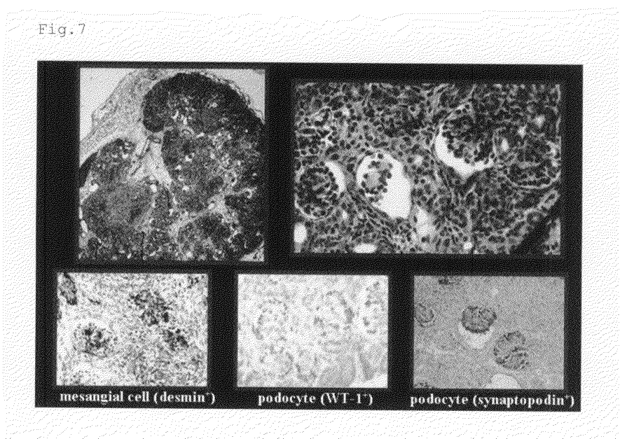

Bioactivity of α-gal A enzyme of the kidney primordial derived from human mesenchymal stem cells, produced by the method described above was evaluated by fluorometry (non-patent document 19). When, as a control, the metanephros of a wild type mouse (left) was compared under the same protocol with that of Fabry mouse (right), the bioactivity of a-gal A in the kidney primordia from the Fabry mouse was extremely low (19.7±5.5 nmol/mg/hour) compared to that from the wild type mouse (655.0±199.6 nmol/mg). However, the kidney primordia having the nephron derived from the injected human mesenchymal stem cells expressed a significantly higher amount of the α-gal A bioactivity (204.2±98.8 nmol/mg/hour, p<0.05, FIG. 5-1(a)) than the wild type mouse.

To confirm the Gb3 clearance capacity of the obtained kidney primordia, an organ culture was carried out in the presence of Gb3, and an analysis was performed by comparing accumulation of Gb3 in the metanephros in the wild type mouse (left) with that in the Fabry mouse (right). It was confirmed that the accumulation of Gb3 in the ureteric bud and S-shaped body in kidney primordial of the Fabry mouse (FIG. 5-2(b) right) was markedly cleared by combining with the nephron derived from the human mesenchymal stem cell, formed by the relay culture method (FIG. 5-2(b) center). This result indicates that the newly produced nephron is functioning biologically.

### F.

The present invention, described up to this point, revealed that allowing hMSCs to grow in a specific organ location in whole-embryo culture can commit them to the fate of the organ. Injection of GDNF-transfected hMSCs into embryos followed by relay culture can create entire nephrons, not just individual kidney cells. These hMCS-derived cells are functional as tested by their ability to metabolize Gb3.

hMSCs can be reprogrammed for other fates and organ structures, depending upon the embryonic environment into which they enter. A further advantage of using hMSCs is that although they are of mesodermal origin, they have the potential to differentiate into cell types that are normally derived from ectoderm or endoderm (non-patent document 23). Therefore, in the present invention the kidney was shown as a representative example, but organs such as the liver and pancreas which are derived from the endodermal germ layer can be reconstituted. Furthermore, specific organs such as an endocrine gland or tissues having a single structure can be generated from autologous MSCs by changing the conditions of the organ culture, after the initiation of organ development and during whole embryo culture.

The host immune system is not yet fully developed at this stage of the whole embryo culture. Therefore, the host is tolerant to foreign cells. The present invention is to establish a method for generating self-organs from autologous MSCs using the endogenous development system of an immunocompromised foreign host.

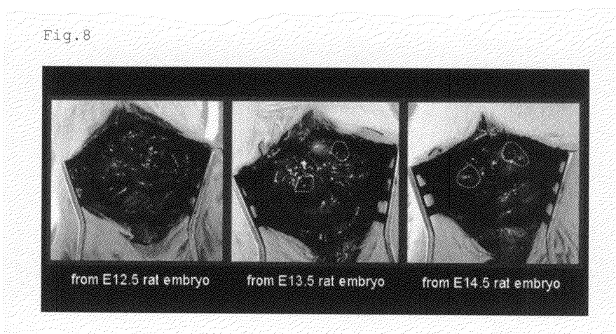

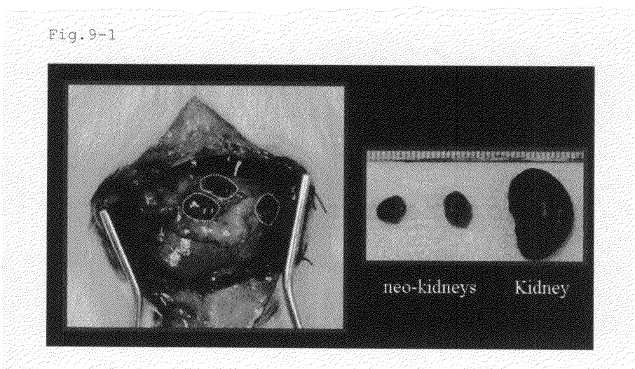

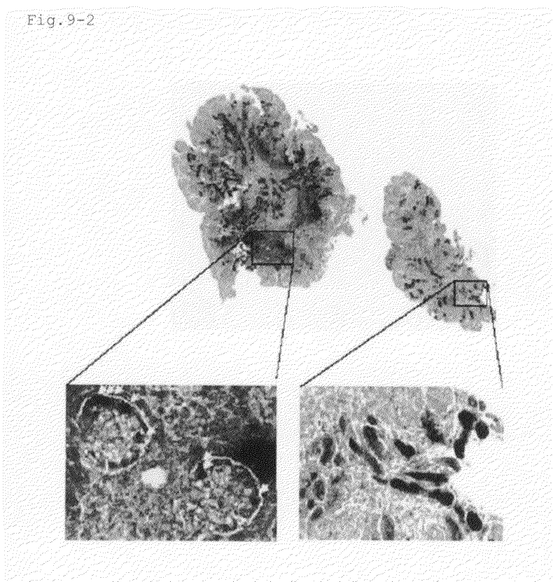

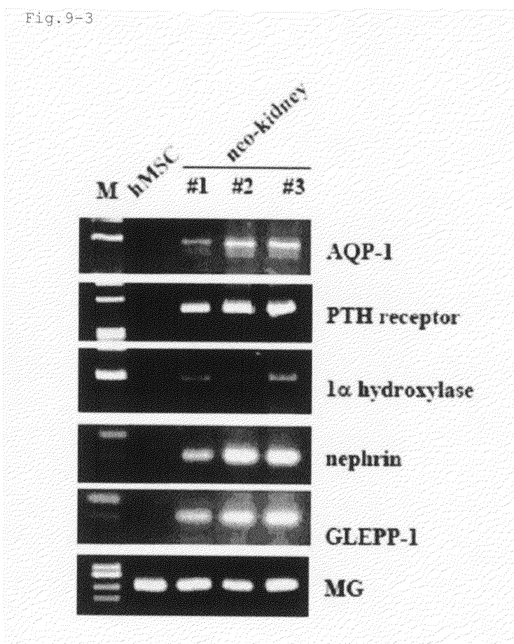

The system described up to this point uses the organ culture for the final growth of the kidney primordia, and therefore the kidney formed does not have blood vessel structure. For this reason, the basic function of the kidney, hemofiltration function can not be confirmed, and therefore the system was further improved. It has been reported that the rat metanephros tissue transplanted to the greater omentum continued growing (non-patent document 24). Thus the metanephros tissue was isolated from the E15 embryo, transplanted to the greater omentum of rat and 2 weeks later laparotomy was performed. It was confirmed that the transplanted metanephros continued to grow further in the greater omentum and the blood vessel system from the greater omentum was invading the kidney (FIG. 6). This growth was not decreased even under kidney failure conditions (after resection of one kidney), but on the contrary it was shown to be further accelerated (FIG. 6). A histological analysis of this grown kidney is shown in FIG. 7. Inside of the kidney, blood vessels are filled with erythrocytes that can not be recognized before the transplantation, showing histologically the opening of the blood circulation. In addition, glomerular mesangial cells (desmin positive) and highly differentiated glomerular epithelium cells (WT-1 and synaptopodin positive cells), which could not be confirmed before the transplantation to the greater omentum, were confirmed. Next, to investigate the best timing for the transplantation, the metanephros at various stages was transplanted to the greater omentum (FIG. 8). As in the figure, it was shown that the transplantation of the premature metanephros tissue up to E12.5 did not induce the growth afterwards, but the kidney grew when the metanephros tissue after E13.5 was transplanted.

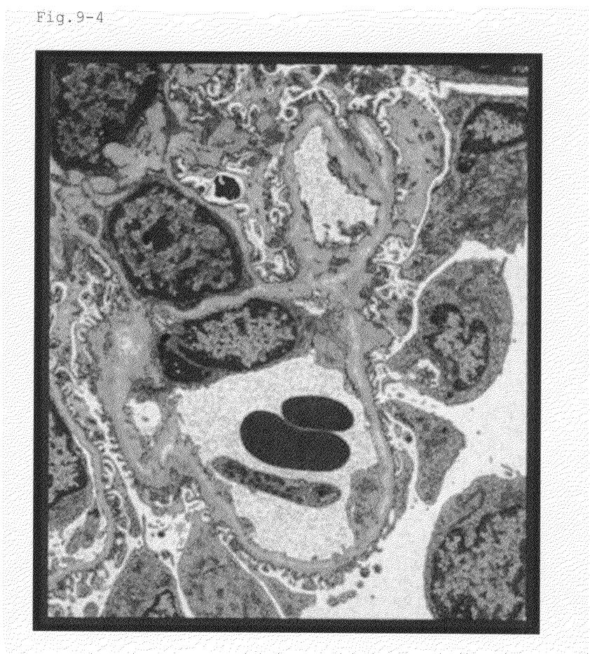

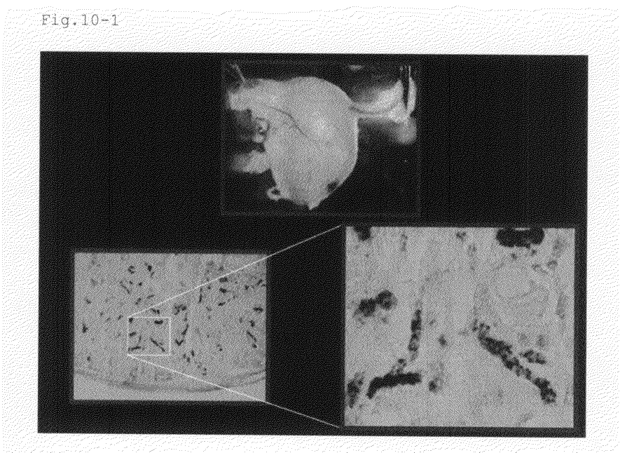

Based on above results, the relay culture method was further improved. That is, after injecting the Lac Z positive GDNF-transfected hMSCs into a rat embryo (E11.5), the whole embryo culture was performed (48 hours), the organ culture was then performed for 24 hours until reaching the stage where a continuous growth in the greater omentum was possible, and then these were transplanted to the greater omentum (referred to the improved relay culture method). To accelerate the growth further one kidney was resected. After 2 weeks, the newly grown kidney reached 64±21 mg (FIG. 9-1). In histological examination using X-gal assay (FIG. 9-2), the LacZ positive hMSCs were morphologically differentiated to the glomerular epithelial cells (Figure below left) and renal tubular epithelial cells (Figure below right). These hMSC-derived LacZ positive cells were separated using FACS-Gal assay, and the gene expression of these was analyzed by RT-PCR to find that the glomerular epithelial cell-specific genes (nephrin and GLEPP-1) and the renal tubular epithelial cell-specific genes (AQP-1, parathyroid hormone (PTH) receptor 1, 1α hydroxylase) were expressed (FIG. 9-3). The electron microscopic analysis confirmed the presence of erythrocytes in the glomerular capillary confirmed the unification with the blood vessel system of the recipient, and in addition, the pedicel of highly differentiated glomerular epithelial cells, and the construction of endothelial cells and mesangial cells were confirmed (FIG. 9-4).

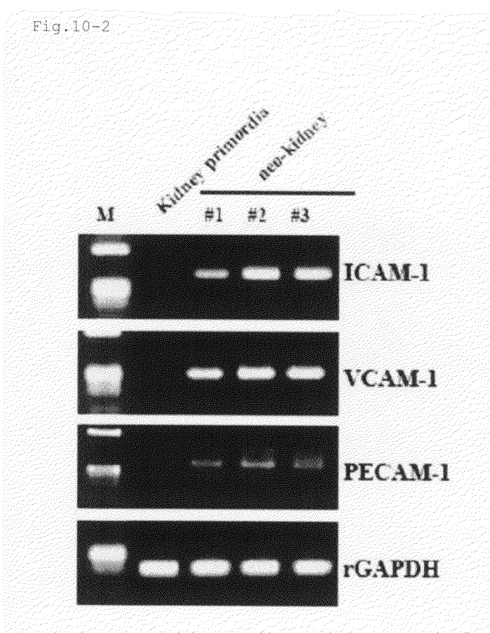

To confirm that this blood was supplied from the blood vessels of the recipient to which transplantation was performed, the kidney primordia was transplanted to the greater omentum of a LacZ rat, the blood vessels of which are stained blue with LacZ. It was shown by macroscopic examination that the blood vessels of the greater omentum were incorporated into the newly formed kidney (FIG. 10-1, upper), and by tissue staining with LacZ, it was demonstrated that the blood vessels in the kidney were formed by the blue cells derived from the recipient (FIG. 10-1, lower). It was confirmed by the RT-PCR of the LacZ positive cells separated by FACS that the LacZ positive cells expressed the vascular endothelial cell-specific genes such as the intercellular adhesion molecule-1 (ICAM-1), vascular cell adhesion molecule-1 (VCAM-1), and platelet-endothelial cell adhesion molecule-1 (PECAM-1) (FIG. 10-2).

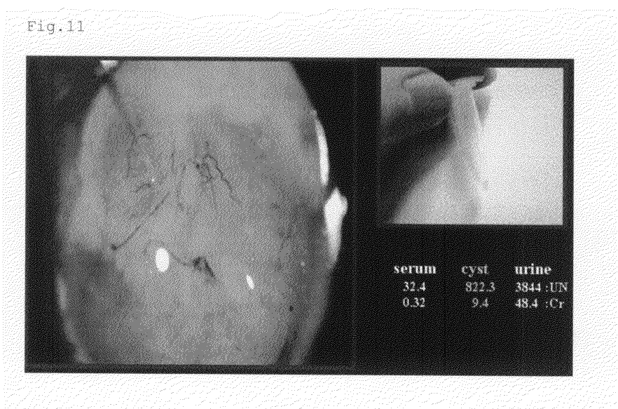

Based on these results, it was examined whether the cloned kidney derived from the human mesenchymal stem cell can produce the recipient's urine by the improved relay culture method. The hMSCs, to which the LacZ and GDNF genes introduced using retrovirus and adenovirus, respectively, were injected to the rat embryo (E11.5) at the kidney forming site. FIG. 11 shows the morphology of the newly formed kidney which was grown for 24 hours in the whole embryo culture and further for 4 weeks in the greater omentum. From the image, it was considered that hydronephrosis was caused by urine produced because there was no opening of the ureter in this kidney. Therefore, the liquid retained in the ureter was recovered to test whether this was urine, and it was found that the composition contained significantly higher concentration of urea-nitrogen and creatinine than that of serum, suggesting that it was urine filtered by the glomerulus. That is, it is effective to form an outlet for urine by treating the ureter of the cloned kidney to make an opening to the recipient's ureter, bladder, rectum or skin between 2 to 4 weeks when the kidney grows and produces urine.

### INDUSTRIAL APPLICABILITY

The present invention allows a new development in organ transplantation, for example, allows a patient, such as a dialysis patient with renal disease, to benefit by the original functions of a freshly generated organ through transplantation of the isolated autologous mesenchymal stem cells to a pregnant host animal to mature into the organ, which is then transplanted to the body of the person.

1-8. (canceled)

9. A method for preparing a desired organ for transplantation to a human by transplanting an isolated human mesenchymal stem cell to an embryo inside a pregnant mammal host or to an embryo dissected from the pregnant mammal host to induce differentiation of said mesenchymal stem cell, wherein said mesenchymal stem cell is transplanted to the embryo at a corresponding site for differentiation into the desired organ in the host at a transplantation time when the host is still on an immunologically tolerant stage.

10. The method according to claim 9, wherein said desired organ is a kidney.

11. The method according to claim 10, wherein the corresponding site for differentiation into said desired organ in the host is a sprouting site of an ureteric bud.

12. The method according to claim 9, wherein said desired organ is a liver, pancreas, lung, heart, cornea, nerve, skin, hematopoietic stem cell or bone marrow.

13. The method according to claim 9, wherein said host is a mammal having a similar size of the organ to that of the desired organ for said human.

14. The method according to claim 9, wherein said host is a pig.

15. The method according to claim 14, wherein said transplantation time is on a stage embryo day of 21 to 35.

16. The method according to claim 9, wherein said mesenchymal stem cell is transplanted to the embryo by transplanting the cell exactly to an organ-forming site of the host through a transuterine approach.

17. The method according to claim 9, wherein whole embryo culture in vitro is further conducted.

18. The method according to claim 17, wherein organ culture in vitro is further conducted.


SEQUENCE LISTING (20 sequences)

SEQ ID NO: 1 - DNA, 20 bp, Artificial
  Synthesis
cctgaacaacgtagtctgcg

SEQ ID NO: 2 - DNA, 20 bp, Artificial
  Synthesis
cagctgtgatctctgagtgg

SEQ ID NO: 3 - DNA, 22 bp, Artificial
  Synthesis
ctggagagcacaaacagcagag

SEQ ID NO: 4 - DNA, 22 bp, Artificial
  Synthesis
aaggccgcagagcaaaagaagc

SEQ ID NO: 5 - DNA, 24 bp, Artificial
  Synthesis
taagttacacagcagtcaaatgga

SEQ ID NO: 6 - DNA, 24 bp, Artificial
  Synthesis
cacatacataaatgccggaatctt

SEQ ID NO: 7 - DNA, 24 bp, Artificial
  Synthesis
agggctcattgcggtggttgtcat

SEQ ID NO: 8 - DNA, 24 bp, Artificial
  Synthesis
taagggtgccttccgttctagagt

SEQ ID NO: 9 - DNA, 24 bp, Artificial
  Synthesis
cttggacacctcctggctattgac

SEQ ID NO: 10 - DNA, 24 bp, Artificial
  Synthesis
agcaggtgggtccctttctttcac

SEQ ID NO: 11 - DNA, 20 bp, Artificial
  Synthesis
gatgcagatgacgtcatgac

SEQ ID NO: 12 - DNA, 21 bp, Artificial
  Synthesis
caggcggtcaaacacctcccg

SEQ ID NO: 13 - DNA, 24 bp, Artificial
  Synthesis
tcactgtggagatgatttcagagg

SEQ ID NO: 14 - DNA, 21 bp, Artificial
  Synthesis
cgtcagcatagttgatccgga

SEQ ID NO: 15 - DNA, 22 bp, Artificial
  Synthesis
caactgggagagactgggagaa

SEQ ID NO: 16 - DNA, 22 bp, Artificial
  Synthesis
aatctgacaacaagacggagca

SEQ ID NO: 17 - DNA, 23 bp, Artificial
  Synthesis
caggtttactcacgtcatccagc

SEQ ID NO: 18 - DNA, 21 bp, Artificial
  Synthesis
tcacatggttcacacggcagg

SEQ ID NO: 19 - DNA, 19 bp, Artificial
  Synthesis
catcaacgaccccttcatt

SEQ ID NO: 20 - DNA, 21 bp, Artificial
  Synthesis
actccacgacatactcagcac